(19) United States
(12) Patent Application Publication
(10) Pub. No.: US 2024/0212839 A1
(43) Pub. Date: Jun. 27, 2024

(54) PSYCHOTHERAPY DEVICE

(71) Applicant: Preston A. Goings, Kent, WA (US)

(72) Inventor: Preston A. Goings, Kent, WA (US)

(21) Appl. No.: 18/368,564

(22) Filed: Sep. 14, 2023

Related U.S. Application Data
(60) Provisional application No. 63/434,947, filed on Dec. 23, 2022.

(51) Int. Cl. G16H 40/67; G10L 15/22; G10L 15/26; G16H 20/70; G16H 50/20

(52) U.S. Cl. CPC G16H 40/67; G10L 15/22; G10L 15/26; G16H 20/70; G16H 50/20; G10L 2015/223

(57) ABSTRACT

Disclosed is a psychotherapy device for providing psychotherapy to a user. The psychotherapy device includes multiple command buttons configured to be pressed by a user, a speaker, and a microphone. A CPU is configured to receive speech signals from the microphone and input signals from the command buttons. The CPU is further configured to transmit speech signals to the speaker. The psychotherapy device acts as a “mental health voice assistant” and the psychotherapy device interacts with the user in a similar way that a psychologist or therapist would do if the user was sitting at the office of the psychologist or therapist.

### CROSS-REFERENCE TO RELATED APPLICATIONS

The present application hereby claims priority to and incorporates by reference the entirety of the disclosures of the provisional application No. 63/434,947, entitled “A PSYCHOTHERAPY DEVICE” filed on 23 Dec. 2022.

### TECHNICAL FIELD

The present invention generally relates to the field of Psychotherapy. More particularly, the present invention relates to a voice-user interface device that provides mental support to various kinds of individual persons.

### BACKGROUND

People around the world suffer from various mental disorders arising due to multiple factors such as but not limited to: homelessness, illness, loneliness, bullying, physical pain/deformities, physical war/battle fights, death of other individuals, lack of money, career, profession and so on. People suffering from such medical disorders require immediate mental support; otherwise, these people could suffer low-quality life, depression, unhappiness, high stress, may commit suicide, and so on. Few people suffering from such medical disorders could also pose a danger to society by committing crimes such as mass shootings, serial murders, torturing other people, rapes, and so on. Presently, many countries such as the United States of America, as well as many communities are facing the mass shooting crisis. In a study titled “Mass Violence in America: Causes, Impacts and Solutions” conducted by the National Council Medical Director Institute, it has been estimated that roughly one-third of acts of mass violence defined as crimes in which four or more people were killed since the 1990s were committed by people with a serious mental illness.

Psychotherapy (also referred to as talking therapy) is the use of psychological methods, particularly based on regular personal interaction, to help a person change behavior, increase happiness, and overcome problems. Psychotherapy aims to improve an individual's well-being and mental health, resolve or mitigate troublesome behaviors, beliefs, compulsions, thoughts, or emotions, and improve relationships and social skills. Combined treatment with psychiatric medication and psychotherapy has become one of the most common modes of psychiatric treatment in current practice.

Psychotherapy may be delivered in person (one on one, with couples, or in groups), over the phone, via telephone counselling, or via the Internet. There have also been developments in computer-assisted therapy, such as virtual reality therapy for behavioral exposure, multimedia programs for each cognitive technique, and handheld devices for improved monitoring or putting ideas into practice. Generally, psychotherapists are mental health professionals such as psychiatrists, psychologists, mental health nurses, clinical social workers, marriage and family therapists, or professional counselors. Psychotherapists usually treat their patients in their clinics, homes, or hospitals.

Generally, it is not possible for various individuals to be treated by psychotherapists due to various reasons such as lack of psychotherapists, costs associated with psychotherapists, visit timings of psychotherapists, geographic locations of psychotherapists, and so on. Nowadays, various psychotherapy devices are being employed for psychotherapy that eliminates some of the problems faced by various individuals who wish to be treated by a psychotherapist.

For instance, WO2012112695A1 discloses methods and systems for online counseling sessions conducted over the Internet. In some configurations, a method for remotely conducting counseling sessions between a client and an expert using an IP-based network includes providing a website accessible to the network wherein the expert and a client both have access to the website. In such configurations, upon accessing and logging into the website, the client is permitted to search a database for online experts and select an appropriate expert for a counseling session. In some configurations, the client initially completes various intake and/or registration forms in a virtual waiting room, wherein such forms are customized per the relevant expert. The method continues as an expert remotely conducts a counseling session with the client via remote means, including video conferencing. Following the counseling session, the client is automatically returned to the virtual waiting room and provided with subsequent counseling-based options.

However, this solution has a few drawbacks. Solutions proposed in WO2012112695A1 require the use of the internet, websites, video cameras and online experts, etc which are not readily accessible every time to an individual user. Further, the solutions proposed in WO2012112695A1 are costly and complicated which could pose a challenge for low income/poor individuals and less tech savvy individual users who are not well-versed with using modern technologies.

The above-mentioned limitations of the psychotherapy devices would restrict the large scale commercial deployment of psychotherapy devices in the market. In an attempt to scale up the use of psychotherapy devices, there remains a need to suggest a simple, easy-to-use psychotherapy device that is capable of solving the aforementioned problems of the psychotherapy devices.

### SUMMARY

Before the present systems and methods, embodiments are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only, and is not intended to limit the scope of the present application. Further, it is also to be understood that the terminology if not defined by the present disclosure should be construed as the general or known meaning/definition of the terminology, known to the person skilled in the art.

It is an object of the present invention to provide a psychotherapy device for providing psychotherapy to a user, the psychotherapy device comprising at least one sidewall, wherein the sidewall(s) comprises a plurality of command buttons configured to be pressed by a user; a top wall and a bottom wall; wherein the sidewall(s), the top wall and the bottom wall are arranged together to define a cavity. The top wall comprises a speaker and at least one microphone; a CPU arranged in the cavity; wherein the CPU is configured to receive speech signals from the microphone and input signals from the command button; and wherein the CPU is configured to transmit speech signals to the speaker.

Embodiments of the present invention disclose a psychotherapy device for providing psychotherapy to a user, wherein the psychotherapy device comprises: at least one sidewall, wherein the sidewalls comprises a plurality of command buttons configured to be manually pressed by a user; wherein each of the command button has a particular command message; a top wall and a bottom wall; wherein the sidewalls, the top wall, and the bottom wall are arranged together to define a cavity; wherein the top wall comprises a speaker and at least one microphone; a CPU arranged in the cavity; wherein the CPU is configured to receive speech signals from the microphones and input signals from the command button; and wherein the CPU is configured to transmit speech signals to the speaker; and a power module configured to provide electric power to the psychotherapy device.

In another embodiment, the sidewalls comprises further comprises an audio-visual camera.

In another embodiment, a swivel base is integrated into at least one of the top wall and/or bottom wall, such that the psychotherapy device rotates relative to the swivel base wherein the axis of rotation of the psychotherapy device corresponds to the longitudinal axis of the swivel base.

In another embodiment, the power module comprises a power cord that extends from at least one of the sidewalls to a power outlet socket; thereby providing electric power to the psychotherapy device.

In another embodiment, the power module includes at least one rechargeable and/or removable battery to provide electric power to the psychotherapy device.

In another embodiment, the power module includes a solar panel to utilize energy from sunlight for electrically powering the psychotherapy device.

In another embodiment, at least one sidewall comprises a display monitor screen to provide a visual display to a user.

In another embodiment, the command messages of the plurality of command buttons are illuminated with an illumination system.

In another embodiment, the psychotherapy device is used at locations selected from group comprising of: homes, offices, railway stations, metro stations, bus stands, homeless shelters, schools, colleges, and retirement homes.

Embodiments of the present invention disclose a psychotherapy device for providing psychotherapy to a user, the psychotherapy device comprises: a user input unit that comprises a plurality of command buttons configured to be manually pressed by user; wherein each of the command button has a particular command message; A speaker module that controls the operation of a speaker; A microphone module that controls the operation of at least one microphone; a CPU electrically connected to all of: the user input unit, the camera unit, the speaker module, the microphone module; and wherein the CPU interacts with the user by selectively operating at least one of: the speaker module, and the microphone module, depending on the command button of the user input unit pressed by user.

In another embodiment, a camera unit controls the operation of an audio-visual camera.

In another embodiment, a communication unit is configured to communicate the psychotherapy device, to a remote device.

In another embodiment, a power module provides electric power to the psychotherapy device.

In another embodiment, a display monitor unit controls a plurality of display monitor screens.

In another embodiment, a memory unit comprises a psychotherapy database comprising multiple statements.

In another embodiment, a cavity at least substantially encloses the speaker module, and the microphone module.

In another embodiment, the CPU comprises a text-to-speech converter for providing speech signals to the speaker module; and the CPU further comprises a speech-to-text converter for processing audio signals from the microphone module.

In another embodiment, the CPU comprises artificial intelligence program that uses machine learning obtained from various user behaviors and/or patterns as well as the Internet; and the artificial intelligence Al program controls the operation of the psychotherapy device accordingly.

In another embodiment, the plurality of command buttons is arranged on at least one sidewall.

Various objects, features, aspects and advantages of the present invention will become more apparent from the following detailed description of the embodiments of the invention, along with the accompanying drawings in which like numerals represent like components.

### BRIEF DESCRIPTION OF THE DRAWINGS

FIG. 1 illustrates a front-perspective view of a psychotherapy device, according to an embodiment of the present invention;

FIG. 2 illustrates a back view of the psychotherapy device of FIG. 1;

FIG. 3 illustrates a schematic block diagram of the psychotherapy device of FIG. 1, according to an embodiment of the present invention; and

FIG. 4 illustrates a front-perspective view of the psychotherapy device, according to another embodiment of the present invention.

### DETAILED DESCRIPTION

Before describing the present invention in detail, it should be observed that the present invention utilizes a combination of components or processes, which constitutes a psychotherapy device. Accordingly, the components or processes have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific component level details and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

References to “one embodiment”, “an embodiment”, “another embodiment”, “one example”, “an example”, “another example” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

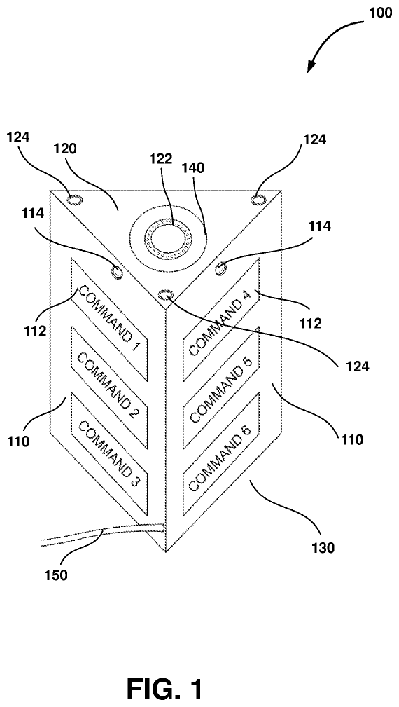

The psychotherapy device will now be described with reference to the accompanying drawings, particularly FIGS. 1-4.

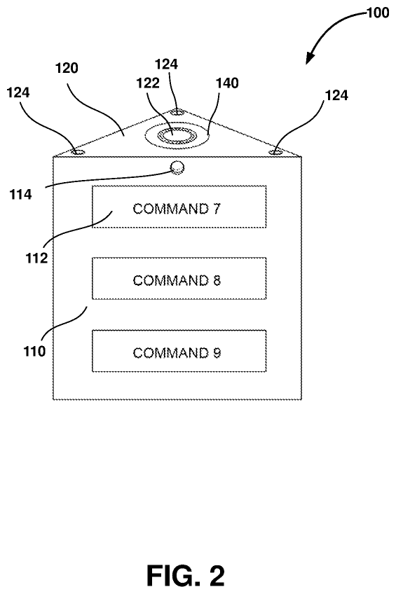

FIG. 1 and FIG. 2 illustrate a front perspective view and a back view of a psychotherapy device 100 respectively that is configured to provide psychotherapy to a user, according to an embodiment of the present invention. The psychotherapy device 100 has three sidewalls 110, a top wall 120 and a bottom wall 130 arranged together to define a cavity 132 (not shown in figures) such that the cross-section of the psychotherapy device 100 is substantially triangle-shaped as shown in FIG. 1. The cavity 132 (not shown in figures) formed by an arrangement of three sidewalls 110, the top wall 120 and the bottom wall 130 comprises/encloses various components/systems that will be described in greater detail in the below description.

Each sidewall 110 comprises a plurality of command buttons 112 and an audio-visual camera 114. In an exemplary embodiment as shown in FIG. 1, each sidewall 110 comprises three command buttons 112 and an audio-visual camera 114, thus a total of nine command buttons 112 in the psychotherapy device 100. The nine command buttons 112 are designated as “COMMAND 1”, “COMMAND 2”, “COMMAND 3”, “COMMAND 4”, “COMMAND 5”, “COMMAND 6”, “COMMAND 7”, “COMMAND 8” and “COMMAND 9”. Further, the top wall 120 comprises a speaker 122 and at least one microphone 124. Further, a swivel base 140 is integrated in at least one of the top wall 120 and/or bottom wall 130 such that the psychotherapy device 100 could rotate relative to the swivel base 140 wherein the axis of rotation of the psychotherapy device 100 corresponds to the longitudinal axis of the swivel base 140. The rotation of the psychotherapy device 100 permits various sidewalls 110 to also rotate relative to the swivel base 140, such that a user can access command buttons 112 of all three sidewalls 110 by staying stationary at a location in close proximity of the psychotherapy device 100 while the psychotherapy device 100 is being rotated. A power cord 150 extends through at least one of the sidewalls 110 to provide electric power to the psychotherapy device 100.

In an exemplary embodiment (not shown in figures), the swivel base 140 is connected to a vertical structure (not shown in figures) connected to a ground surface (not shown in figures) wherein vertical structure (not shown in figures) could include but not limited to: pole, tube, rod, pillar and so on.

In an exemplary embodiment (not shown in figures), each sidewall 110 comprises a display monitor screen (not shown in figures) to provide a visual display such as a picture or a video to a user. Thus, a total of three display monitor screens (not shown in the figures) are arranged (installed) in the psychotherapy device 100.

Each of the command buttons 112 has a particular command message displayed on the command button 112 in at least one language used by the majority of the community. The command message displayed on each command button 112 is distinct from one another such that “COMMAND 1”, “COMMAND 2”, “COMMAND 3”, “COMMAND 4”, “COMMAND 5”, “COMMAND 6”, “COMMAND 7”, “COMMAND 8” and “COMMAND 9” are distinct from each other. The command message of each command button 112 represents a particular mood/feeling of a user. The mood/feeling could include but is not limited to: anger, sadness, de-motivation, boredom, stress, frustration, anxiety, guilt, feeling bullied, needing help, suicidal thoughts, and so on.

In an exemplary embodiment as shown in FIG. 1, nine command buttons 112 have a total of nine command messages displayed on three sidewalls 110 such that each sidewall 110 has a total of three command messages. On the first sidewall 110 of the three sidewalls 110, the command messages are (a) I want to kill (b) Revenge someone (c) I am being bullied. On the second sidewall 110 of the three sidewalls 110, the command messages are (a) I want to die (b) I want to talk (c) Tell me a story. On the third sidewall 110 of the three sidewalls 110, the command messages are (a) How to enjoy life (b) Connect to a therapist (c) Assistance. However, it should be understood that the nine command messages of are exemplary only and the command message could include any other sentence/question such as but not limited to: entertain me, sing a relaxing song, tell an inspirational quote and so on

In an exemplary embodiment, the command messages displayed are in a combination of at least two languages, wherein one language is English language and the other language is Braille script (language) such that the command message displayed in the English language is displayed on the top half section of a command button 112, and the command message written in Braille script (language) is written (inscribed) on the bottom half section of a command button 112. In another embodiment (not shown in figures), the language could include other languages including but not limited to: French, German, Russian, Hindi, Mandarin (Chinese), Japanese, Korean and so on. In an exemplary embodiment, the command messages displayed are numerical (numbers) such as but not limited to: one, two, three, four and so on.

In an exemplary embodiment, the command messages of the plurality of command buttons 112 are illuminated with an illumination system (not shown in figures) such that a user can visually see the command messages written in command buttons 112 in severe dark conditions. The illumination system (not shown in figures) could include but not be limited to: back-lit LED (light emitting diode), LASER (light amplification by the stimulated emission of radiation), and light bulb and so on. In an embodiment, each command button 112 is illuminated with a back-lit LED (not shown in figures).

In an exemplary embodiment, the command messages of command buttons 112 can be reprogrammed (changed) depending on the usage environment of the psychotherapy device 100, to allow the same psychotherapy device 100 to be used at various locations such as but not limited to: homes, offices, railway stations, metro stations, bus stands, homeless shelters, schools, colleges, retirement homes and so on. Thus, the command messages of command buttons 112 can be costumed by either changing the reading language of the command message or by changing the entire context (meaning) of the command message to suit the environment of the psychotherapy device 100.

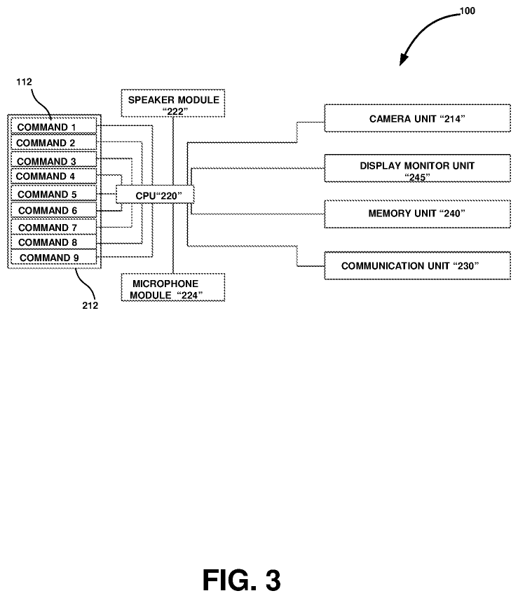

FIG. 3 illustrates a schematic block diagram (flow chart) of a psychotherapy device 100 of FIG. 1, according to an embodiment of the present invention. As shown in FIG. 3, the block diagram (control logic chart) of the psychotherapy device 100 comprises a plurality of command buttons 112 (collectively referred as “a user input unit” 212) arranged on at least one sidewall 110, a CPU (“central processing unit”) 220, a camera unit 214, a speaker module 222, a microphone module 224, a communication unit 230, a memory unit 240, a display monitor unit 245 and a power module (not shown in figures). The camera unit 214 controls the operation of the audio-visual camera 114, the speaker module 222 controls the operation of speaker 122, and the microphone module 224 controls the operation of the microphone(s) 124. The communication unit 230 is configured to communicate the psychotherapy device 100 to a remote device (not shown in figures) and the power module (not shown in figures) provides electric power to all the required components/systems of the psychotherapy device 100, the display monitor unit 245 (not shown in figures) controls a plurality of display monitor screens (not shown in figures). The cavity 132 (not shown in figures) at least substantially encloses the communication unit 230, memory unit 240, power module (not shown in figures), the speaker module 222, and the microphone module 224.

The CPU 220 is central brain (main controller) of the psychotherapy device 100, and the CPU 220 is electrically connected to all of the: a plurality of command buttons 112 (collectively referred as “user input unit” 212), the camera unit 214, the speaker module 222, the microphone module 224, the communication unit 230, the memory unit 240, a display monitor unit 245 (not shown in figures) and the power module (not shown in figures). In an embodiment, the CPU 220 comprises a text-to-speech converter (not shown in figures) for providing speech signals to the speaker module 222. In an embodiment, the CPU 220 further comprises a speech-to-text converter (not shown in figures) for processing audio signals from the microphone module 224. In an embodiment, the CPU 220 comprises an artificial intelligence (AI) program, that uses machine learning obtained from various user behaviors and/or patterns as well as the Internet and then, the artificial intelligence (AI) program controls the operation of the psychotherapy device 100 accordingly.

In an embodiment (not shown in figures), the communication unit 230 utilizes a network to connect to a remote device (not shown in figures), wherein the network includes but not limited to: internet, WIFI (wireless LAN), Bluetooth, Radio-frequency waves, mobile phone network, Telephone Hotline, cable wiring, satellite network, Ethernet, broadband and so on. The remote device (not shown in figures) could include but is not limited to: mobile phone, laptop, landline telephone, computer, transceiver, satellite phone, walkie-talkie, computer, television, medical doctor's device, psychotherapist's device, and emergency hotlines such as 911 and so on.

In an embodiment (not shown in figures), the memory unit 240 comprises a psychotherapy database (not shown in figures) comprising multiple statements/algorithms. The statements/algorithms could be in the form of questions, answers, stories, jokes, motivational quotes, pictures, videos, songs, and dialogues. The psychotherapy database (not shown in the figures) could be generated with the assistance of various psychotherapists and various books/articles/research papers of psychotherapy. In an embodiment, the psychotherapy database (not shown in the figures) could be accessed by inserting a USB (universal serial bus) stick or memory card in the memory unit 240.

In another embodiment, the psychotherapy database (not shown in figures) is hard-wired in the memory unit 240 of the psychotherapy device 100 such that the psychotherapy database is installed in the memory unit 240 of the psychotherapy device 100 at the time of manufacturing of the psychotherapy device 100. Further, the psychotherapy database (not shown in figures) could be updated online by using networks such as but not limited to: Internet, WIFI (wireless LAN), Bluetooth, radio communication, and so on.

In an embodiment (not shown in figures), the power module (not shown in figures) provides power to all the required components/systems of the psychotherapy device 100 such as but not limited to: a user input unit 212, a CPU 220, a camera unit 214, a speaker module 222, a microphone module 224, a communication unit 230, a memory unit 240, and the display monitor unit 245. The power module (not shown in figures) could include necessary electrical components required for providing electric power such as but not limited to: resistors, transformers, wires, inverters, rectifiers, PCB, micro-controllers, and so on. In an embodiment as shown in FIG. 1, the power module (not shown in figures) includes a power cord 150 that electrically connects to a power outlet socket (not shown in figures) to provide electric power to the psychotherapy device 100.

In an embodiment (not shown in figures), the power module (not shown in figures) is electrically connected to each one of: a user input unit 212, a CPU 220, a camera unit 214, a speaker module 222, a microphone module 224, a communication unit 230, a memory unit 240, and the display monitor unit 245 for providing electric power.

An exemplary method of using the psychotherapy device 100 will now be described in reference to FIGS. 1-3. A user manually presses a command button 112 after reading the command message written on the command button 112. Depressing (pressing) the command button 112 triggers an electric signal that is fed into a CPU 220. The CPU 220 selectively operates at least one of: the camera unit 214, the speaker module 222, the microphone module 224, the communication unit 230, the memory unit 240, the display monitor unit 245 (not shown in figures) depending on the command button 112.

In an exemplary embodiment, when the user presses the “COMMAND 1” command button 112, then the CPU 220 selectively operates the microphone module 224 such that, the user can speak to the psychotherapy device 100 using the microphone(s) 124 of the microphone module 224. Afterward, the user's speech is converted into an electrical speech signal by the microphone module 224 and then the speech signal is fed into the CPU 220. The CPU 220 processes (converts) the speech signal into text signal using memory unit 240 and/or the CPU 220 itself, and based on the processed text signal, the CPU 220 operates at least one of: a camera unit 214, a speaker module 222, a microphone module 224, a communication unit 230, and the display monitor unit 245. Afterward, the psychotherapy device 100, in particular CPU 220, would interact with the user by doing either one of the following:—(a) providing questions to the user by using the speaker module 222 and/or (b) listening to the user by using the microphone module 224 and/or (c) displaying information for the eyes of the user by using the display monitor unit 245 (not shown in figures) and/or (d) gathering video/photo of the user by using the camera unit 214 (e) transmitting user's information to the remote device (not shown in figures) by using the communication unit 230. Thus, the psychotherapy device 100 interacts with the user in a similar way, a psychologist or therapist would do if the user was sitting at the office of the psychologist or therapist.

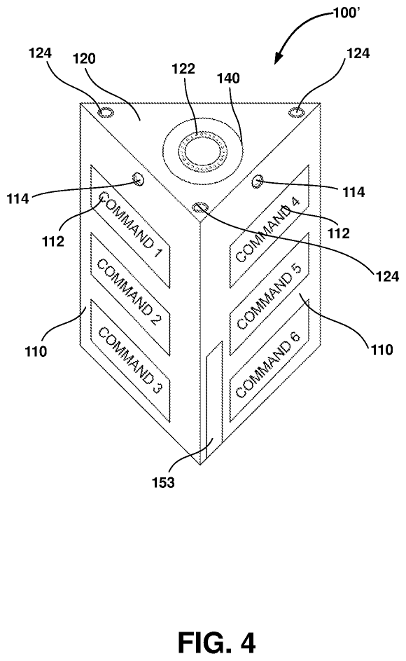

FIG. 4 illustrates a front perspective view of a psychotherapy device 100′, according to another embodiment of the present invention. The psychotherapy device 100′ is similar to the psychotherapy device 100′ of FIGS. 1-2, except with the modifications associated with the power module (not shown in figures). As shown in FIG. 4, the power module (not shown in figures) includes at least one rechargeable and/or removable battery 153 to provide electric power to the psychotherapy device 100′. The rechargeable and/or removable battery 153 acts as a power source such that an external power outlet is not always required for powering the psychotherapy device 100′. Thus, the psychotherapy device 100′ could be used at locations where electricity is not readily accessible.

In another embodiment (not shown in figures), the power module (not shown in figures) includes a solar panel (not shown in figures) to utilize energy from sunlight for electrically powering the psychotherapy device 100. Thus, the psychotherapy device 100′ could be used at locations where electricity is not readily accessible.

The psychotherapy device (100, 100′) of FIGS. 1-4 has three sidewalls 110. However, it should be understood that the psychotherapy device (100, 100′) could have any arbitrary number of sidewalls 110 such as but not limited to: one, two, four, five, and six, and so on. Thus, the psychotherapy device (100, 100′) could effectively function with any number of sidewalls 110 with little to no variation in the functionality of the psychotherapy device (100, 100′).

The psychotherapy device (100, 100′) of FIGS. 1-4 comprises a plurality of command buttons 112 (collectively referred to as “user input unit” 212), a CPU 220, a camera unit 214, a speaker module 222, a microphone module 224, a communication unit 230, a memory unit 240, a display monitor unit 245 and a power module (not shown in figures).

However, it should be understood that the psychotherapy device (100, 100′) could also effectively function without using some of the components/systems described above. For instance, in another embodiment (not shown in figures), a psychotherapy device 100″ (not shown in figures) comprises only a plurality of command buttons 112 (collectively referred to as “user input unit” 212), a CPU 220, a speaker module 222, a microphone module 224 and a power module (not shown in figures) similar to FIGS. 1-4. However, the psychotherapy device 100″ (not shown in figures) does not comprise the camera unit 214, the communication unit 230, the memory unit 240, and the display monitor unit 245. The Psychotherapy device 100″ (not shown in figures) comprises at least one sidewall 110, wherein the sidewalls 110 comprises a plurality of command buttons 112 configured to be pressed by a user; a top wall 120 and a bottom wall 130; wherein the sidewalls, the top wall 120 and the bottom wall 130 are arranged together to define a cavity 130; wherein the top wall 120 comprises a speaker 122 and at least one microphone 124. A CPU 220 is arranged in the cavity 130; wherein the CPU 220 is configured to receive speech signals from the microphone(s) 124 and input signals from the command button 112; and wherein the CPU 220 is configured to transmit speech signals to the speaker 122. The advantage of psychotherapy device 100″ of this particular embodiment is that it is simpler in design, easy to use, easy to operate, and requires fewer components and thus has minimal cost.

Certain advantages of the psychotherapy device (100, 100′, 100″) of the present disclosure are listed herein below:—
The psychotherapy device (100, 100′, 100″) interacts with the user similarly to what a psychologist or therapist would do if the user was sitting at the office of the psychologist or therapist. Thus, the psychotherapy device (100, 100′, 100″) could act as a “mental health voice assistant”.
The psychotherapy device (100, 100′, 100″) reduces the compulsive violent actions of users. For example, a person doesn't have to suppress their angry feelings because a psychotherapy device (100, 100′, 100″) will be with them at their home/their location talking to them and encouraging them to express their feelings.
The psychotherapy device (100, 100′, 100″) can be used for various user ages of adolescence, middle adulthood, and senior citizens.
The installation of the psychotherapy device (100, 100′, 100″) could be done at various locations such as but not limited to: homes, offices, railway stations, metro-stations, bus stands, homeless shelters, schools, colleges, retirement homes and so on, thus providing psychotherapy help to a variety of users.
Further, the psychotherapy device (100, 100′, 100″) could be installed in booths such that homeless people could be able to talk to the psychotherapy device (100, 100′, 100″) at any time for free.

The various components and parts of the various embodiments of the psychotherapy device (100, 100′, 100″) of the present invention are similar and interchangeable. It is obvious to the one skilled in the art that the various components, and parts of the psychotherapy device (100, 100′, 100″) of one embodiment of the present invention could be considered for other embodiments with little or no variation.

It should be understood according to the preceding description of the present invention that the same is susceptible to changes, modifications, and adaptations, and that the said changes, modifications, and adaptations fall within the scope of the appended claims.

What is claimed is:

1. A psychotherapy device (100, 100′, 100″) for providing psychotherapy to a user, the psychotherapy device (100, 100′, 100″), comprising:
at least one sidewall (110), wherein the sidewall(s) (110) comprises a plurality of command buttons (112) configured to be manually pressed by a user; wherein each of the command buttons (112) has a particular command message;
a top wall (120) and a bottom wall (130);
wherein the sidewall(s), the top wall (120), and the bottom wall (130) are arranged together to define a cavity (130);
wherein the top wall (120) comprises a speaker (122) and at least one microphone (124); a CPU (220) arranged in the cavity (130); wherein the CPU (220) is configured to receive speech signals from the microphone(s) (124) and input signals from the command button (112); and wherein the CPU (220) is configured to transmit speech signals to the speaker (122); and
a power module configured to provide electric power to the psychotherapy device (100, 100′, 100″).

2. The psychotherapy device (100, 100′, 100″) according to claim 1, wherein the sidewall(s) (110) comprises further comprises an audio-visual camera (114).

3. The psychotherapy device (100, 100′, 100″) according to claim 1, wherein a swivel base (140) is integrated in at least one of the top wall (120) and/or bottom wall (130), such that the psychotherapy device (100) rotates relative to the swivel base (140) wherein the axis of rotation of the psychotherapy device (100) corresponds to the longitudinal axis of the swivel base (140).

4. The psychotherapy device (100, 100′, 100″) according to claim 1, wherein the power module comprises a power cord (150) that extends from at least one of the sidewall(s) (110) to a power outlet socket; thereby providing electric power to the psychotherapy device (100, 100′, 100″).

5. The psychotherapy device (100, 100′, 100″) according to claim 1, wherein the power module includes at least one rechargeable and/or removable battery (153) to provide electric power to the psychotherapy device (100, 100′, 100″).

6. The psychotherapy device (100, 100′, 100″) according to claim 1, wherein the power module includes a solar panel to utilize energy from sunlight for electrically powering the psychotherapy device (100, 100′, 100″).

7. The psychotherapy device (100, 100′, 100″) according to claim 1, wherein at least one sidewall (110) comprises a display monitor screen to provide a visual display to a user.

8. The psychotherapy device (100, 100′, 100″) according to claim 1, wherein the command messages of the plurality of command buttons (112) are illuminated with an illumination system.

9. The psychotherapy device (100, 100′, 100″) according to claim 1, wherein the psychotherapy device (100, 100′, 100″) is used at locations selected from a group comprising of: homes, offices, railway stations, metro stations, bus stands, homeless shelters, schools, colleges, and retirement homes.

10. The psychotherapy device (100, 100′, 100″) for providing psychotherapy to a user, the psychotherapy device (100, 100′, 100″), comprising:
a user input unit (212) that comprises a plurality of command buttons (112) configured to be manually pressed by a user; wherein each of the command buttons (112) has a particular command message;
a speaker module (222) that controls the operation of a speaker (122);
a microphone module (224) that controls the operation of at least one microphone (124);
a CPU (220) electrically connected to all of: the user input unit (212), the camera unit (214), the speaker module (222), the microphone module (224); and
wherein the CPU (220) interacts with the user by selectively operating at least one of: the speaker module (222), and/or the microphone module (224), depending on the command button (112) of the user input unit (212) pressed by the user.

11. The psychotherapy device (100, 100′, 100″) according to claim 10, wherein a camera unit (214) controls the operation of an audio-visual camera (114).

12. The psychotherapy device (100, 100′, 100″) according to claim 10, wherein a communication unit (230) is configured to communicate the psychotherapy device (100, 100′) to a remote device.

13. The psychotherapy device (100, 100′, 100″) according to claim 10, wherein a power module provides electric power to the psychotherapy device (100, 100′).

14. The psychotherapy device (100, 100′, 100″) according to claim 10, wherein a display monitor unit (245) controls a plurality of display monitor screens.

15. The psychotherapy device (100, 100′, 100″) according to claim 10, wherein a memory unit (240) comprises a psychotherapy database comprising multiple statements.

16. The psychotherapy device (100, 100′, 100″) according to claim 10, wherein a cavity (132) at least substantially encloses the speaker module (222), and the microphone module (224).

17. The psychotherapy device (100, 100′, 100″) according to claim 10, wherein the CPU (220) comprises a text-to-speech converter for providing speech signals to the speaker module (222), and the CPU (220) further comprises a speech-to-text converter for processing audio signals from the microphone module (224).

18. The psychotherapy device (100, 100′, 100″) according to claim 10, wherein the CPU (220) comprises an artificial intelligence program that uses machine learning obtained from various user behaviors and/or patterns as well as the Internet; and the artificial intelligence (AI) program controls the operation of the psychotherapy device (100).

19. The psychotherapy device (100, 100′, 100″) according to claim 10, wherein the plurality of command buttons (112) are arranged on at least one sidewall (110).
